(19) United States
(12) Patent Application Publication
(10) Pub. No.: US 2021/0407683 A1
(43) Pub. Date: Dec. 30, 2021

(54) METHOD AND SYSTEM FOR REMOTE HEALTH MONITORING, ANALYZING, AND RESPONSE

(71) Applicant: Verizon Patent and Licensing Inc., Basking Ridge, NJ (US)

(72) Inventors: Bhumip Khasnabish, Lexington, MA (US); Rohit Shirish Saraf, Nashua, NH (US)

(21) Appl. No.: 16/916,946

(22) Filed: Jun. 30, 2020

(51) Int. Cl. G16H 50/30; G16H 40/67; G16H 50/20; A61B 5/0205

(52) U.S. Cl. CPC G16H 50/30; A61B 5/02055; G16H 50/20; G16H 40/67

(57) ABSTRACT

A method, a system, and a non-transitory storage medium are described in which a health monitoring, analyzing, and response service is provided. The service may ingest monitoring information from end devices. The service may assign an adaptive weight value to each instance of monitoring information based on profile information pertaining to the end device. The profile information may include operational characteristics information and rating information. The service may aggregate the monitoring information based on the adaptive weight values to produce a weighted health or state value. The service may analyze and determine a health or condition of a person or object. The service may also generate response information reactive to the determined health or condition.

### BACKGROUND

Remote monitoring of health relating to a person or a thing has significantly advanced in recent years. Various sensing devices may be used to collect data regarding the health or state of a person or a thing.

### BRIEF DESCRIPTION OF THE DRAWINGS

FIG. 1 is a diagram illustrating an exemplary environment in which an exemplary embodiment of a health monitoring, analyzing, and response service may be implemented;

FIG. 2A is a diagram illustrating exemplary components of an exemplary embodiment of the ingestion device depicted in FIG. 1;

FIG. 2B is a diagram illustrating an exemplary process of an aggregation service;

FIG. 2C is a diagram illustrating another exemplary process of the aggregation service;

FIG. 3 is a diagram illustrating an exemplary process of an analyzing service;

FIG. 4 is a diagram illustrating exemplary components of a device that may correspond to one or more of the devices illustrated and described herein; and

FIG. 5 is a flow diagram illustrating an exemplary process of an exemplary embodiment of the health monitoring, analyzing, and response service.

### DETAILED DESCRIPTION OF PREFERRED EMBODIMENTS

The following detailed description refers to the accompanying drawings. The same reference numbers in different drawings may identify the same or similar elements. Also, the following detailed description does not limit the invention.

Technologies that support remote monitoring of a living thing (e.g., a human being, an animal, etc.) or objects have advanced over recent years. For example, a health monitoring device, such as a sensor, may collect data relating to an individual's blood pressure, blood sugar, oxygen level, temperature, and/or other types of health metrics. The data may be recorded and/or monitored for evaluation. According to other examples, a monitoring device may collect data relating to manufacturing, industrial machinery, utilities, or other business-related processes and operations. This data may also be recorded and/or monitored for evaluation regarding the health or state of a device or a thing, or aspects of manufacturing, production, a process flow, and so forth.

Manual inspection of such data, however, may be prone to errors. Additionally, the use of disparate monitoring devices, applications, and/or services may not provide coordinated and trustworthy data results. For example, different monitoring devices may provide different measured values for the same parameter. Additionally, different monitoring devices may offer different levels of precision, accuracy, reliability, or another metric. Additionally, in-person visits by a patient to a doctor, a hospital, or other medical entity may be time-consuming, costly, and may be risky in various circumstances, particular when the person or the entity may not be easily and/or readily accessible for inspection and/or treatment.

According to exemplary embodiments, a health monitoring, analyzing, and response service is described. According to an exemplary embodiment, the health monitoring, analyzing, and response service may include an ingestion service. The ingestion service may support the receipt of data pertaining to different applications and monitoring devices associated with a person or a thing. The ingestion service may allow for the management of different applications and/or monitoring devices, such as the addition of new applications and/or monitoring devices or the deletion of existing applications and/or services, to be used by health monitoring, analyzing, and response service.

According to an exemplary embodiment, the health monitoring, analyzing, and response service may include an aggregation service. The aggregation service may generate and assign a weight value for an application based on profile information relating to the application. For example, the profile information may relate to the functions, interfaces, capabilities, and/or other types of operational characteristics of the application, as described herein. The profile information may also include other types of information, such as rating information (e.g., ratings by users, an industry, medical community, or other sources), an accuracy value, a trustworthiness value, and/or other types of relevant information. The weight value may be within a numerical range of values.

The aggregation service may generate and assign a weight value for a monitoring device, such as a sensor or other device, based on profile information. The profile information may similarly relate to functions, interfaces, capabilities, and/or other types of operational characteristics of the monitoring device, and other types of relevant information, as described herein.

According to an exemplary embodiment, the health monitoring, analyzing, and response service may include an analyzing service. For example, the analyzing service may obtain data from the aggregation service and analyze the data. The analyzing service may generate response information based on the vital or other type of metric data pertaining to a person or a thing subject to the service. For example, the response information may include a pre-diagnosis, a recommendation, an augment to a diagnosis, or other corrective action.

According to an exemplary embodiment, the health monitoring, analyzing, and response service may include a response service. The response service may provide or make available the response information to the monitoring device, as described herein.

In view of the foregoing, the health monitoring, analyzing, and response service may improve the confidence and reliability of data relating to remote health monitoring service. For example, the accuracy and confidence in data stemming from various applications and sensors may not be equal. The health monitoring, analyzing, and response service may manage an array of different applications and sensing devices that may be used and prioritize the data based on the profile information to enable healthcare professionals or other entities to confidently act on the recommendations, pre-diagnosis, or other corrective actions provided.

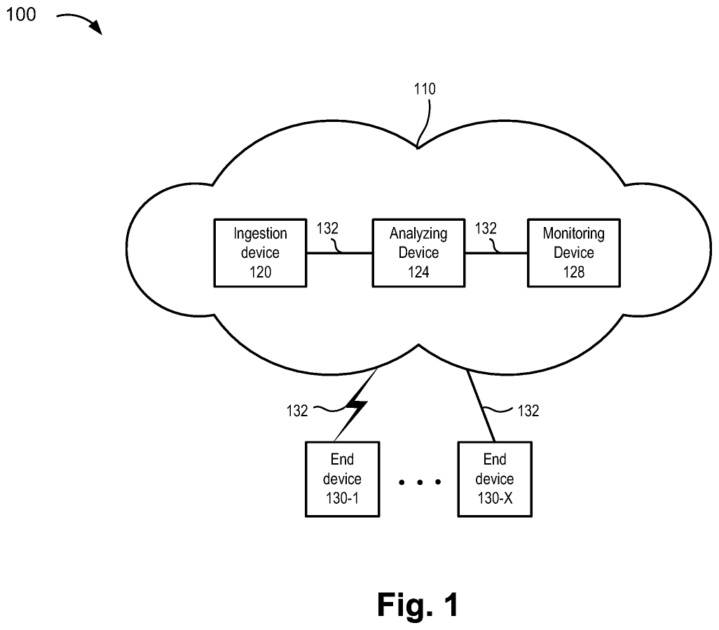

FIG. 1 is a diagram illustrating an exemplary environment 100 in which an exemplary embodiment of the health monitoring, analyzing, and response service may be implemented. As illustrated, environment 100 includes a network 110. Network 110 may include an ingestion device 120, an analyzing device 124, and a monitoring device 128. Environment 100 further includes end devices 130-1 through 130-X (referred to as end devices 130, or individually or generally as end device 130).

The number, the type, and the arrangement of network devices in network 110, as illustrated and described, are exemplary. The number of end devices 130 is exemplary. A network device, a network element, or a network function (referred to herein simply as a network device) may be implemented according to one or multiple network architectures (e.g., a client device, a server device, a peer device, a proxy device, a cloud device, a virtualized function, and/or another type of network architecture (e.g., Software Defined Networking (SDN), virtual, logical, network slicing, etc.)). Additionally, a network device may be implemented according to various computing architectures, such as centralized, distributed, cloud (e.g., elastic, public, private, etc.), edge, fog, and/or another type of computing architecture.

Environment 100 includes communication links 132 between network devices, and between end devices 130 and network 110. Environment 100 may be implemented to include wired, optical, and/or wireless communication links. A communicative connection via communication link 132 may be direct or indirect. For example, an indirect communicative connection may involve an intermediary device and/or an intermediary network not illustrated in FIG. 1. A direct communicative connection may not involve an intermediary device and/or an intermediary network. The number and the arrangement of communication links 132 illustrated in environment 100 are exemplary.

Network 110 may include one or multiple networks of one or multiple types of technologies. For example, network 110 may include an access network, a core network, a fronthaul network, a backhaul network, an application layer network, a private network, a public network, a wide area network (WAN), a local area network (LAN), a service provider network, a municipal area network (MAN), a wireless network, a wired network, an optical network, a virtualized network, a multi-access edge computing (MEC) network, a data center, a cloud network, a packet-switched network, and/or another type of communication network.

Ingestion device 120 may receive data pertaining to different applications and end devices 130 associated with the monitoring of a person or a thing. Ingestion device 120 may allow for the management of different applications and/or monitoring devices, such as the addition of new applications and/or monitoring devices or the deletion of existing applications and/or services, to be used by health monitoring, analyzing, and response service. Ingestion device 120 may also aggregate the received data based on the aggregation service, as described herein.

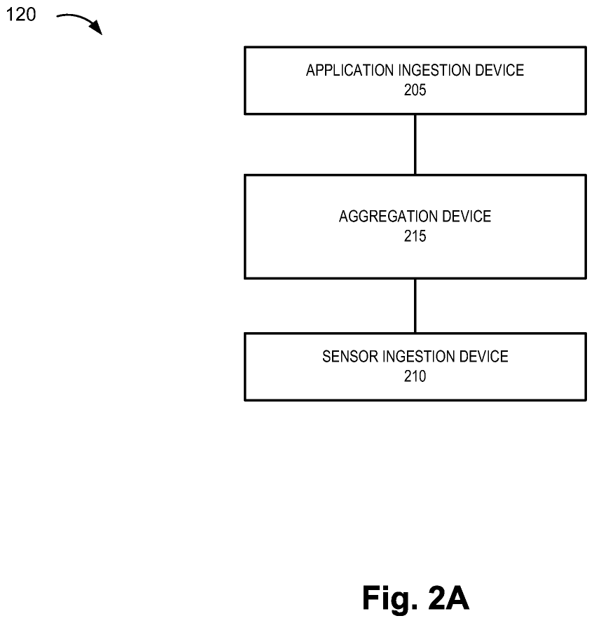

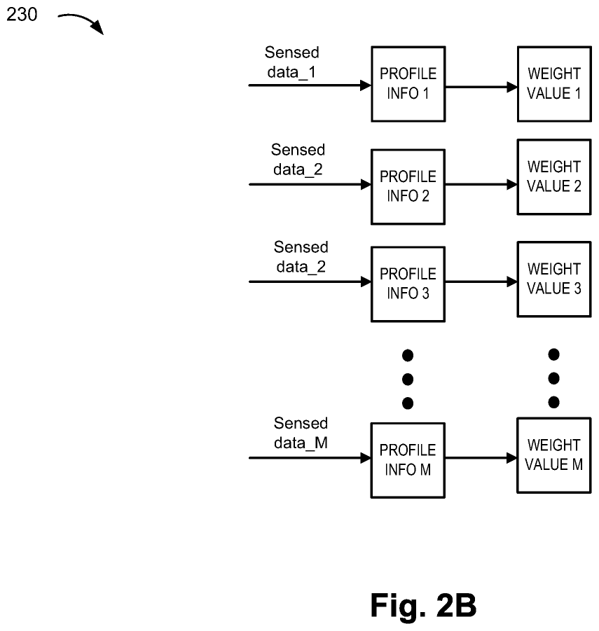

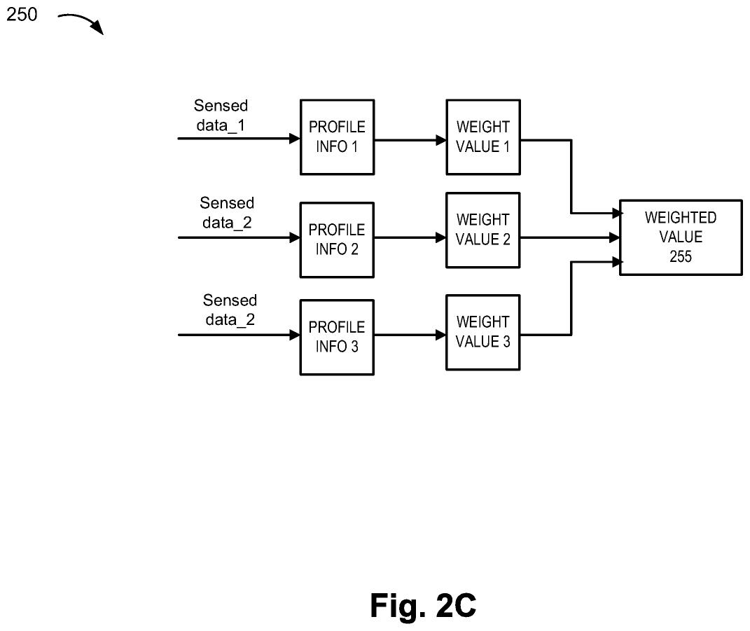

Ingestion device 120 may generate and assign an adaptive weight value for the received data based on profile information relating to the application. Ingestion device 120 may also generate an adaptive weight value for the received data based on profile information relating to the sensor device. The sum of all the weights may be normalized (e.g., to 1) Ingestion device 120 is described further below in relation to FIGS. 2A-2C. Ingestion device 120 may aggregate the data based on the received data and weights, as described herein.

FIG. 2A is a diagram illustrating components of an exemplary embodiment of ingestion device 120. As illustrated, ingestion device 120 may include an application ingestion device 205, a sensor ingestion device 210, and an aggregation device 215. According to other exemplary embodiments, ingestion device 120 may include additional, fewer, or different types of devices that provide the ingestion and aggregation services, as described herein. Additionally, or alternatively, multiple of the exemplary components may be implemented as a single device, or an exemplary component may be implemented as multiple devices.

Application ingestion device 205 may include an interface that receives data from various types of applications. The interface may support various communication protocols (e.g., Internet Protocol (IP), HTTP, TCP, etc.) and various types of traffic (e.g., intermittent, streaming or continuous, batch, etc.). The interface may also support security measures (e.g., encryption, authentication, authorization, etc.) and other communication-related criteria to ensure proper receipt of the data from the application or other source. These and other types of configuration may be addressed during the on-boarding of the application to the health monitoring, analyzing, and response service.

For example, a smartphone may include an application that communicates data relating to the monitoring of a person. Alternatively, a server device (e.g., a third party server, etc., not shown) may receive data from the application via end device 130, and the server device has an application programming interface (API) to transmit the data to application ingestion device 205. As an example, a wearable device may include an application that transmits sensed or application data relating to vitals of the user to a server device. The server device may store, analyze, and/or provide some other service relating to the sensed or application data. The server device may further forward the sensed or application data (e.g., raw or processed) to application ingestion device 205 in accordance with permissions from the user.

Sensor ingestion device 210 may include an interface that receives data from various types of sensor devices. Similar to application ingestion device 205, sensor ingestion device 210 may support various communication protocols, types of traffic, security measures, and other communication-related criteria to ensure proper receipt of the data. Additionally, a sensor device may transmit data to an intermediary device (e.g., a server), and the server may provide the sensed data to sensor ingestion device 210 via an API and in accordance with permissions from the user or subject of end device 130 (e.g., an application, a sensor device, a mobile device, etc.).

Aggregation device 215 may identify data that relates to the same end device 130, such as an application and/or a sensor device. Aggregation device 215 may also identify data that relates to the same subject (e.g., a person, an object or thing) of the monitoring.

Aggregation device 215 may assign a weight value to an instance of data (e.g., a packet of data, a stream of data, etc.). The weight value may be a static value or a dynamic value (e.g., an adaptive weight value). For example, the weight value may change based on updated or new profile information. According to another example, the weight value may (automatically) change (e.g., lower the weight value) based on the age of the end device (e.g., sensor of the end device, etc.) which may cause a degradation in the confidence of the data. The decremental value may configured relative to a time period (e.g., per year, etc.). Additionally, the decremental value may be determined on other factors, such as the type of traffic (e.g., continuous versus intermittent) and associated usage may contribute to the calculation of the decremental value. Conversely, information indicating an update to an existing application or sensor device may form the basis to change the weight value (e.g., increase). As such, aggregation device 215 may re-calculate the weight value. The weight value may be generated based on profile information for end device 130 (e.g., an application or a sensor device). For example, for a sensor device, the profile information may relate to the functions, interfaces, capabilities, and/or other types of operational characteristics. By way of further example, the profile information may include features of the sensor device, such as triggers (e.g., inputs), hardware/software characteristics (e.g., processor, memory, graphics, etc.), ingress/egress frequency bands, data types, interfaces, type of sensed data (e.g., audio, video, tactile, heart rate, temperature, respiration, etc.), type of sensor (e.g., wearable, swallowable, digital, analog, active, passive, contact-type, non-contact type, temperature sensor, heart rate sensor, respiration rate sensor, low-cost, specialized, ambient, decoration, furniture, etc.), power, heat, noise, and/or vibrational characteristics, and/or other types of operational or configuration attributes of the sensor device. The features of the sensor device may be obtained based on the specification of the sensor device, independent testing, certifications, or other sources (e.g., the vendor, public information, etc.), for example. The profile information may also include other types of information, such as rating information from various sources (e.g., users, an expert, an industry, medical community, or other sources), an accuracy value, a trustworthiness value, and/or other types of relevant information that may indicate a measure of the quality of the sensor device and/or the sensed data.

Additionally, for example, for an application, the profile information may include features of the application, such as input and output functions, technology and interface plus usage, logic usage information, errors and exception conditions, and/or other types of operational or configuration attributes of the application. For example, the applications may operate on various end devices 130 (e.g., applications for Apple® device, applications for Android® devices, etc.) and may use a peripheral device (e.g., a sensor device) for obtaining a vital (e.g., temperature, glucose reading, etc.) of a user. Alternatively, end device 130 may include the application and a sensor device (e.g., a wearable device, etc.). The features of the application may be obtained based on independent testing or other sources (e.g., the vendor, public information, etc.), for example. The profile information may also include other types of information, such as rating information from various sources (e.g., users, an expert, an industry, medical community, or other sources), an accuracy value, a trustworthiness value, and/or other types of relevant information that may indicate a measure of the quality of the application or the application (sensed) data.

The weight value may be within a numerical range of values, such as 0.01 to 0.99 or some other range of low and high values. The weight value may indicate a level of confidence, accuracy, trustworthiness, precision, or other measure of quality of the sensed data. For example, assume the numerical range is 1 to 10, and a sensor device or an application is be assigned a value of 10. The sensed data may be given greater weight relative to data from another sensor device or another application that may be assigned a value of 4 for purposes of analyzing the sensed data, diagnosing, and/or other response.

FIG. 2B is a diagram illustrating an exemplary process 230 of aggregation device 215. As illustrated, sensed data from end devices 130-1 through M (e.g., applications and/or sensor devices) may be received by aggregation device 215. Aggregation device 215 may apply profile information pertaining to the application and/or sensor device to calculate a weight value for each sensed data. According to various exemplary scenarios, one or more of the weight values 1-M may be the same, or each weight value may be different.

FIG. 2C is a diagram illustrating another exemplary process 250 of aggregation device 215. The number of sensed data (e.g., 1-3) is merely exemplary. According to this example, sensed data 1-3 may relate to the same person or thing and the same vital (e.g., temperature, etc.) or state/metric. As illustrated, aggregation device 215 may calculate a weighted value 255 for the vital based on weight values 1-3 associated with sensed data 1-3. For example, weighted value 255 may be a weighted average value for the vital pertaining to the sensed data 1-3. By way of further example, assume that sensed data 1-3 relates to temperature and are from different end devices 130. Sensed data_1 may indicate a temperature of 98 degrees and may have a weight value_1 of 0.2, sensed data_2 may indicate a temperature of 99 degrees and may have a weight value_2 of 0.8, and sensed data_3 may indicate a temperature of 98.2 degrees and may have a weight value_3 of 0.5. The weighted average value for temperature may be 98.6 degrees.

In this way, aggregation device 215 may provide resiliency in the vital or state/metric values and may improve the confidence and trustworthiness in this data for the analytics and response services. Additionally, aggregation device 215 allows readings from different applications and sensor devices for the same (or closely related) vitals or states to be collaboratively used.

Referring back to FIG. 1, analyzing device 124 may include obtaining data from the aggregation service and analyzing the data. Analyzing device 124 may generate response information based on the analysis, as described herein. For example, the response information may include a pre-diagnosis, an augment to a diagnosis, a recommendation, or other responsive action.

According to various exemplary embodiments, the analysis of the data may include various types of procedures, such as trend analysis, comparison to threshold values, single parameter evaluation, multi-parameter evaluation, and other types of anomaly or diagnostic detection procedures. For example, for a trend analysis procedure, analyzing device 124 may evaluate values relating to a vital (e.g., temperature, heart rate, or other type of health parameter) or other type of metric based on historical data associated with the person or object to identify a trend. The identification of the trend may relate to one or multiple values over a time period and the behavior of such values, such as increasing, decreasing, steady-state, and so forth. Analyzing device 124 may also compare the values to various threshold values that may correlate to various conditions, such as the presence or worsening of a health condition, contraction of a virus or a disease, or other state or performance metric relating to the person or thing.

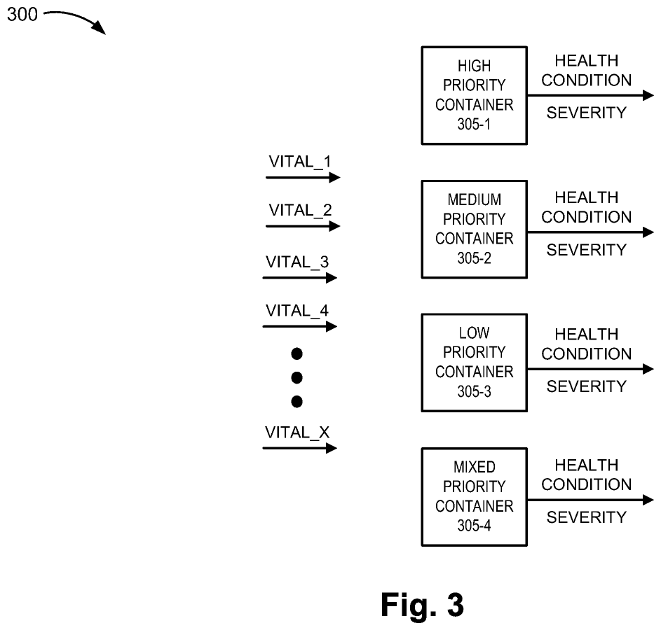

According to an exemplary embodiment, analyzing device 124 may include multiple buckets or containers relating to a particular person, a thing, or a situation. For example, referring to FIG. 3, according to an exemplary process 300, various vitals (e.g., 1−X) may relate to a person subscribed to the health monitoring, analyzing, and response service. The vitals 1-X may relate to the monitoring of one or multiple known chronic conditions. According to other examples, this may not be the case. Each of the vitals 1-X may include a weighted value, as calculated in relation to the aggregation service.

As further illustrated, containers 305-1 through 305-4 (also referred to as containers 305, or generally or individually as container 305) may be configured for detection of a condition or a state, and a level or severity of the condition, based on one or multiple vitals 1-X. For example, according to this example, high priority container 305-1 may detect a condition of a high priority or severity level, medium priority container 305-2 may detect a condition of a medium priority or severity level, low priority container 305-3 may detect a condition of a low priority or severity level, and mixed priority container 305-4 may detect a condition that may include multiple levels of priority or severity levels. The number of containers 305 and the type of containers (e.g., high, medium, etc.) are exemplary.

Containers 305 may include logic that may be configured to receive certain vitals and evaluate the values based on threshold values, as well as other factors, such as trend behavior, time periods, and so forth. According to various exemplary embodiments, the threshold values may be personalized to the person or object, and/or non-personalized (e.g., generic or macro). For example, in the case of a pandemic or other disease, illness, etc., situation, containers 305 may include a personalized threshold value for a person with a pre-existing condition or other aspects associated with their overall health profile for detecting the spread of the pandemic, disease, illness, and/or relating to the pre-existing condition. By way of further example, the personalized threshold value may include blood pressure, blood sugar level, cancerous cells growth, or other health condition. Additionally, or alternatively, containers 305 may include non-personalized thresholds that may be used (generally) for a given locale and persons of interest. For example, the macro threshold value may include temperature, respiration, or other health metric or vital. Analyzing device 124 may evaluate the values based on the type of threshold values applied.

The vitals received by each container 305 may include a single vital or multiple and different vitals. As an example, the vitals for high priority container 305-1 may relate to heart rate and/or respiration, the vitals for medium priority container 305-2 may relate to temperature and/or blood pressure, the vitals for low priority container 305-3 may relate to body aches and/or sleeplessness, and the vitals for mixed priority container 305-4 may relate to digestion issues and/or anxiety. Based on the analytics of containers 305, analyzing device 124 may identify different types of health conditions and different severity levels of the health conditions. Additionally, containers 305 may calculate a confidence value or other type of value for the health condition based on the weighted value associated with each vital analyzed.

Referring back to FIG. 1, as mentioned, analyzing device 124 may generate response information based on the analysis of the data received by the aggregation service and processed through ingestion device 120. According to various exemplary embodiments, analyzing device 124 may provide the response information to monitoring device 128. Depending on the circumstances, the response information may include a pre-diagnosis, a recommendation, or another type of corrective action. For example, the response information may warn the person and/or the person's physician about the health condition, recommend taking medication, to undergo frequent observation, quarantine, move to emergency care, or check into a hospital. According to other examples, the response information may include a request to dispatch an ambulance, a request to initiate a diagnosis or a review by a physician, medical expert, or other medical entity (e.g., a virtual clinician or physician, etc.), a trigger for an alarm, or a report or information to populate a report (e.g., an Open Electronic Health Record (EHR) or other suitable electronic medical record), based on prior authorization and/or requirements.

Given the sensitivity of the response information, the transmission of the response information to monitoring device 128 would be authorized by the person subject to the monitoring or other authorized person. In this way, analyzing device 124 may provide the response information to certain monitoring devices 128 and/or persons. Additionally, analyzing device 124 would implement various security measures relating to the response information. For example, Health Insurance Portability and Accountability Act (HIPAA) requirements, other types of guidelines relating to security and/or confidentiality of personally identifiable information (PII), such as the National Institute of Standards and Technology (NIST), as well as local (e.g., city, county, etc.), state, and federal laws that may apply to the response information may be implemented.

According to some exemplary embodiments, the response information may be augmented. For example, the response information may include other data, such as audio, video (e.g., texture, color, time lapse, etc.), text, and/or other types of relevant information as annotations. For example, the augmentation of the response information may be performed by analyzing device 124 and/or monitoring device 128.

Monitoring device 128 may include a network device to receive the response information from analyzing device 124. For example, private data stores, servers, edge or near-edge data centers which may be hosted by community healthcare centers, town health/human services office, a primary care physician's office, a hospital, or other authorized entity may be implemented as monitoring device 128. According to other examples, a privately controlled data vault or a private vital data bank (PVDB) may receive the response information based on real-time authentication, secure network path setup, and encryption information transfer using batch, stream, transaction, etc. based communications. Additionally, for example, other local, zonal, community, etc., based servers may be identified and used for (temporary) monitoring vitals for authorizing access to social gatherings, public events, travel using public transportation, and the like. As previously mentioned, monitoring device 128 may also relate to an ambulance service, a health record repository (e.g., EHR, etc.), and other facilities (e.g., labs, pharmacy, etc.) or types of destinations (e.g., virtual physician, dashboards, etc.). Also, monitoring device 128 may support tracing of a health condition of an individual or a community, and subsequent decisions or recommendations under various scenarios.

End device 130 includes a device that has communicative capabilities. Depending on the implementation, end device 130 may be a mobile device, a portable device, a stationary device, a device operated by a user, or a device not operated by a user. For example, end device 130 may be implemented as a smartphone, a mobile phone, a computer, a pen, a notebook, a tablet, a wearable device (e.g., a watch, wrist band, ankle band, waistband, headband, neckband, necklace, armband, or other type of wearable device), or other suitable device. End device 130 may include various types of software (e.g., applications, programs, etc.) relating to (health) monitoring. The number and the types of software may vary among end devices 130.

According to various exemplary embodiments, end device 130 may include a sensor. For example, a wearable device may include one or multiple sensors. Alternatively, various usable devices, such as a chair, a desk, a bed, a table, or other type of object may include a sensor. Additionally, or alternatively, other types of things may include sensors, such as a decoration in a room, in a wall, or other location for monitoring a person or a thing, a swallowable device, and so forth. Further, end device 130 may be implemented as any number or type of sensors that may monitor a thing or process in relation to manufacturing, etc., as previously mentioned.

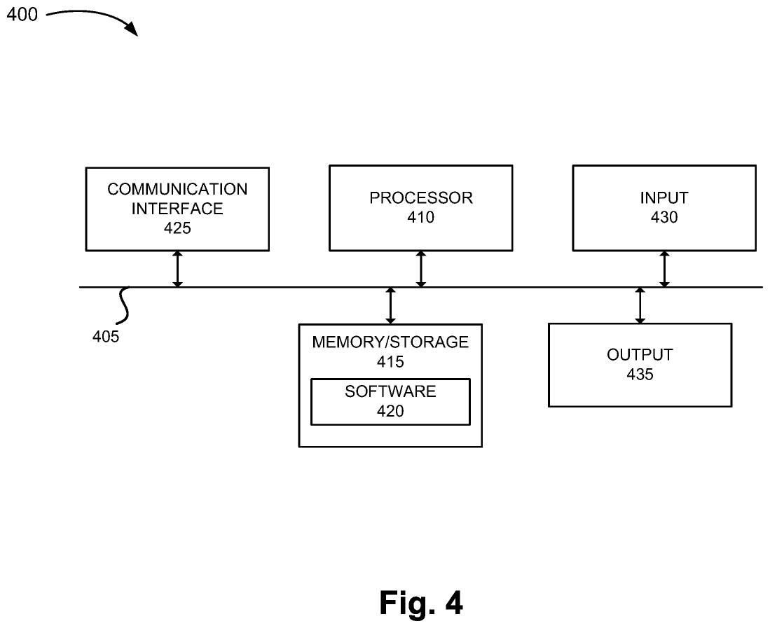

FIG. 4 is a diagram illustrating exemplary components of a device 400 that may be included in one or more of the devices described herein. For example, device 400 may be included in ingestion device 120, analyzing device 124, monitoring device 128, end device 130, application ingestion device 205, sensor ingestion device 210, aggregation device 215, and other types of network devices or logic, as described herein. As illustrated in FIG. 4, device 400 includes a bus 405, a processor 410, a memory/storage 415 that stores software 420, a communication interface 425, an input 430, and an output 435. According to other embodiments, device 400 may include fewer components, additional components, different components, and/or a different arrangement of components than those illustrated in FIG. 4 and described herein.

Bus 405 includes a path that permits communication among the components of device 400. For example, bus 405 may include a system bus, an address bus, a data bus, and/or a control bus. Bus 405 may also include bus drivers, bus arbiters, bus interfaces, clocks, and so forth.

Processor 410 includes one or multiple processors, microprocessors, data processors, co-processors, graphics processing units (GPUs), application specific integrated circuits (ASICs), controllers, programmable logic devices, chipsets, field-programmable gate arrays (FPGAs), application specific instruction-set processors (ASIPs), system-on-chips (SoCs), central processing units (CPUs) (e.g., one or multiple cores), microcontrollers, neural processing unit (NPUs), and/or some other type of component that interprets and/or executes instructions and/or data. Processor 410 may be implemented as hardware (e.g., a microprocessor, etc.), a combination of hardware and software (e.g., a SoC, an ASIC, etc.), may include one or multiple memories (e.g., cache, etc.), etc.

Processor 410 may control the overall operation or a portion of operation(s) performed by device 400. Processor 410 may perform one or multiple operations based on an operating system and/or various applications or computer programs (e.g., software 420). Processor 410 may access instructions from memory/storage 415, from other components of device 400, and/or from a source external to device 400 (e.g., a network, another device, etc.). Processor 410 may perform an operation and/or a process based on various techniques including, for example, multithreading, parallel processing, pipelining, interleaving, etc.

Memory/storage 415 includes one or multiple memories and/or one or multiple other types of storage mediums. For example, memory/storage 415 may include one or multiple types of memories, such as, a random access memory (RAM), a dynamic random access memory (DRAM), a static random access memory (SRAM), a cache, a read only memory (ROM), a programmable read only memory (PROM), an erasable PROM (EPROM), an electrically EPROM (EEPROM), a single in-line memory module (SIMM), a dual in-line memory module (DIMM), a flash memory (e.g., 2D, 3D, NOR, NAND, etc.), a solid state memory, and/or some other type of memory. Memory/storage 415 may include a hard disk (e.g., a magnetic disk, an optical disk, a magneto-optic disk, a solid state disk, etc.), a Micro-Electromechanical System (MEMS)-based storage medium, and/or a nanotechnology-based storage medium. Memory/storage 415 may include drives for reading from and writing to the storage medium.

Memory/storage 415 may be external to and/or removable from device 400, such as, for example, a Universal Serial Bus (USB) memory stick, a dongle, a hard disk, mass storage, off-line (e.g., private, mobile, hybrid, cloud, etc.) storage, or some other type of storing medium (e.g., a compact disk (CD), a digital versatile disk (DVD), a Blu-Ray disk (BD), etc.). Memory/storage 415 may store data, software, and/or instructions related to the operation of device 400.

Software 420 includes an application or a program that provides a function and/or a process. As an example, with reference to ingestion device 120 and analyzing device 124, software 420 may include an application that, when executed by processor 410, provides a function of the health monitoring, analyzing, and response service, as described herein. Software 420 may also include firmware, middleware, microcode, hardware description language (HDL), and/or other form of instruction. Software 420 may also be virtualized. Software 420 may further include an operating system (OS) (e.g., Windows, Linux, Android, proprietary, etc.).

Communication interface 425 permits device 400 to communicate with other devices, networks, systems, and/or the like. Communication interface 425 includes one or multiple wireless interfaces and/or wired interfaces. For example, communication interface 425 may include one or multiple transmitters and receivers, or transceivers. Communication interface 425 may operate according to a protocol stack and a communication standard. Communication interface 425 may include an antenna. Communication interface 425 may include various processing logic or circuitry (e.g., multiplexing/de-multiplexing, filtering, amplifying, converting, error correction, API, etc.). Communication interface 425 may be implemented as a point-to-point interface, a service based interface, etc., as previously described.

Input 430 permits an input into device 400. For example, input 430 may include a keyboard, a mouse, a display, a touchscreen, a touchless screen, a button, a switch, an input port, speech recognition logic, and/or some other type of visual, auditory, tactile, etc., input component. Output 435 permits an output from device 400. For example, output 435 may include a speaker, a display, a touchscreen, a touchless screen, a light, an output port, and/or some other type of visual, auditory, tactile, etc., output component.

Device 400 may perform a process and/or a function, as described herein, in response to processor 410 executing software 420 stored by memory/storage 415. By way of example, instructions may be read into memory/storage 415 from another memory/storage 415 (not shown) or read from another device (not shown) via communication interface 425. The instructions stored by memory/storage 415 cause processor 410 to perform a process and/or a function, as described herein. Alternatively, for example, according to other implementations, device 400 performs a process and/or a function as described herein based on the execution of hardware (processor 410, etc.).

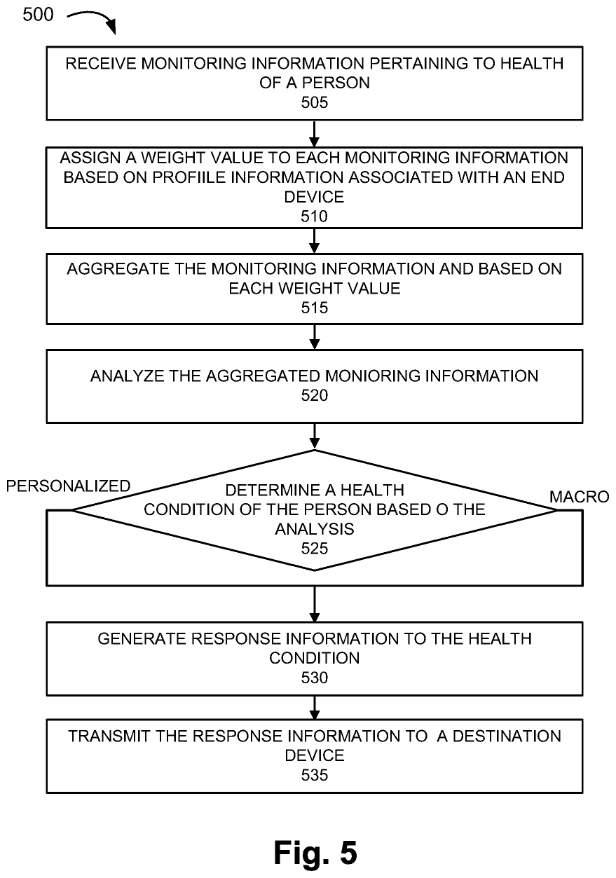

FIG. 5 is a flow diagram illustrating an exemplary process 500 of an exemplary embodiment of the health monitoring, analyzing, and response service. According to an exemplary embodiment, ingestion device 120 and analyzing device 124 may perform steps of process 500. According to an exemplary implementation, processor 410 may execute software 420 to perform a step illustrated in FIG. 5 and described herein. Alternatively, a step illustrated in FIG. 5 and described herein, may be performed by execution of only hardware. For purposes of description of process 500, ingestion device 120 and analyzing device 124 are referred to as a health system.

Referring to FIG. 5, in block 505, the health system may receive monitoring information pertaining to the health of a person. For example, the health system may receive sensed data and/or application data relating to the monitoring of the health of the person from multiple end devices 130 (e.g., applications, sensor devices, or both). The monitoring information may include one or multiple values over one or multiple time periods relating to the same or different vitals or other health metrics.

In block 510, the health system may assign a weight value monitoring information based on profile information associated with end device 130. For example, the health system may store the profile information that indicates characteristics of the end device 130, as described herein. The health system may assign the weight value to the monitoring information associated with end device 130 (e.g., an application, a sensor device, or both) based on the profile information.

In block 515, the health system may aggregate the monitoring information based on each weight value. For example, the health system may aggregate the monitoring information that relate to the same vital or other type of health parameter. For example, the aggregated monitoring information may indicate a weighted average temperature value or a blood pressure value (e.g., single value or range of values).

In block 520, the health system may analyze the aggregated monitoring information. For example, the health system may use trend analysis, comparisons to threshold values, the containers, and/or other techniques for determining the health condition of the person, as described herein.

In block 525, the health system may determine a health condition of the person based on the analysis. For example, the health system may identify the health condition and a severity level of the health condition, as described herein. The health system may select one or multiples personalized threshold values and/or one or multiple macro threshold values to use for identifying the health condition. For example, depending on the person being monitored and the person's medical history, the health system may select a personalized threshold value that may differ from a corresponding macro threshold value. Additionally, for example, the health system may select a macro threshold value based on the geographic area of where the person is situated, demographic information, or other factors. According to some exemplary embodiments, for purposes of trend analysis, the health system may apply a macro threshold value.

In block 530, the health system may generate response information to the health condition. For example, the response information may include a pre-diagnosis, an augment to a diagnosis, a recommendation, or another type of corrective action, as described herein.

In block 535, the health system may transmit the response information to a destination device. For example, the health system may transmit or the response information to various monitoring devices, as described herein.

FIG. 5 illustrates an exemplary process 500 of the health monitoring, analyzing, and response service, however, according to other embodiments, process 500 may include additional operations and/or different operations than those illustrated in FIG. 5 and described herein. For example, process 500 may include annotating the response information. Additionally, for example, according to other exemplary embodiments, the monitoring information may relate to the state or condition of an object, a process, or other type of living thing (e.g., an animal, etc.), and/or the health condition may relate to an anomaly, a performance metric, or the like. According to other exemplary embodiments, the monitoring information may relate to a single (instead of multiple end devices 130).

As set forth in this description and illustrated by the drawings, reference is made to “an exemplary embodiment,” “an embodiment,” “embodiments,” etc., which may include a particular feature, structure or characteristic in connection with an embodiment(s). However, the use of the phrase or term “an embodiment,” “embodiments,” etc., in various places in the specification does not necessarily refer to all embodiments described, nor does it necessarily refer to the same embodiment, nor are separate or alternative embodiments necessarily mutually exclusive of other embodiment(s). The same applies to the term “implementation,” “implementations,” etc.

The foregoing description of embodiments provides illustration but is not intended to be exhaustive or to limit the embodiments to the precise form disclosed. Accordingly, modifications to the embodiments described herein may be possible. For example, various modifications and changes may be made thereto, and additional embodiments may be implemented, without departing from the broader scope of the invention as set forth in the claims that follow. The description and drawings are accordingly to be regarded as illustrative rather than restrictive.

The terms “a,” “an,” and “the” are intended to be interpreted to include one or more items. Further, the phrase “based on” is intended to be interpreted as “based, at least in part, on,” unless explicitly stated otherwise. The term “and/or” is intended to be interpreted to include any and all combinations of one or more of the associated items. The word “exemplary” is used herein to mean “serving as an example.” Any embodiment or implementation described as “exemplary” is not necessarily to be construed as preferred or advantageous over other embodiments or implementations.

In addition, while a series of blocks has been described regarding the process illustrated in FIG. 5, the order of the blocks may be modified according to other embodiments. Further, non-dependent blocks may be performed in parallel. Additionally, other processes described in this description may be modified and/or non-dependent operations may be performed in parallel.

Embodiments described herein may be implemented in many different forms of software executed by hardware. For example, a process or a function may be implemented as “logic,” a “component,” or an “element.” The logic, the component, or the element, may include, for example, hardware (e.g., processor 410, etc.), or a combination of hardware and software (e.g., software 420).

Embodiments have been described without reference to the specific software code because the software code can be designed to implement the embodiments based on the description herein and commercially available software design environments and/or languages. For example, various types of programming languages including, for example, a compiled language, an interpreted language, a declarative language, or a procedural language may be implemented.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements.

Additionally, embodiments described herein may be implemented as a non-transitory computer-readable storage medium that stores data and/or information, such as instructions, program code, a data structure, a program module, an application, a script, or other known or conventional form suitable for use in a computing environment. The program code, instructions, application, etc., is readable and executable by a processor (e.g., processor 410) of a device. A non-transitory storage medium includes one or more of the storage mediums described in relation to memory/storage 415. The non-transitory computer-readable storage medium may be implemented in a centralized, distributed, or logical division that may include a single physical memory device or multiple physical memory devices spread across one or multiple network devices.

To the extent the aforementioned embodiments collect, store or employ personal information of individuals, it should be understood that such information shall be collected, stored, and used in accordance with all applicable laws concerning protection of personal information. Additionally, the collection, storage and use of such information can be subject to consent of the individual to such activity, for example, through well known “opt-in” or “opt-out” processes as can be appropriate for the situation and type of information. Collection, storage and use of personal information can be in an appropriately secure manner reflective of the type of information, for example, through various encryption and anonymization techniques for particularly sensitive information.

No element, act, or instruction set forth in this description should be construed as critical or essential to the embodiments described herein unless explicitly indicated as such.

All structural and functional equivalents to the elements of the various aspects set forth in this disclosure that are known or later come to be known are expressly incorporated herein by reference and are intended to be encompassed by the claims.

What is claimed is:

1. A method comprising:
receiving, by a network device from end devices, monitoring information pertaining to a person, wherein the monitoring information relates to a same health metric;
assigning, by the network device, an adaptive weight value to each monitoring information associated with each of the end devices based on profile information for each of the end devices;
aggregating, by the network device, each monitoring information based on the adaptive weight value associated with each of the end devices;
analyzing, by the network device, the aggregated monitoring information;
determining, by the network device based on the analyzing, a health condition of the person;
generating, by the network device, response information that relates to the health condition; and
transmitting, by the network device, the response information to a destination device.

2. The method of claim 1, wherein the profile information includes data indicating operational characteristics and rating information of the end devices.

3. The method of claim 1, wherein the adaptive weight value indicates a level of quality of each monitoring information.

4. The method of claim 1, wherein the aggregating comprises:
calculating, by the network device, a weighted average of the same health metric based on the adaptive weight value associated with each monitoring information.

5. The method of claim 1, wherein the analyzing comprises:
comparing, by the network device, the aggregated monitoring information to a threshold value.

6. The method of claim 1, wherein the determining comprises:
determining, by the network device, a severity level of the health condition.

7. The method of claim 1, further comprising:
annotating, by the network device, the response information with at least one of audio, video, or text.

8. The method of claim 1, wherein the end devices include a sensor device of a first end device and an application of a second end device.

9. A network device comprising:
a processor configured to:
receive from end devices, monitoring information pertaining to a person, wherein the monitoring information relates to a same health metric;
assign an adaptive weight value to each monitoring information associated with each of the end devices based on profile information for each of the end devices;
aggregate each monitoring information based on the adaptive weight value associated with each of the end devices;
analyze the aggregated monitoring information;
determine, based on the analysis, a health condition of the person;
generate response information that relates to the health condition; and
transmit the response information to a destination device.

10. The network device of claim 9, wherein the profile information includes data indicating operational characteristics and rating information of the end devices.

11. The network device of claim 9, wherein the adaptive weight value indicates a level of quality of each monitoring information.

12. The network device of claim 9, wherein, when aggregating, the processor is further configured to:
calculate a weighted average of the same health metric based on the adaptive weight value associated with each monitoring information.

13. The network device of claim 9, wherein when analyzing, the processor is further configured to:
compare the aggregated monitoring information to a threshold value.

14. The network device of claim 9, wherein the processor is further configured to:
annotate the response information with at least one of audio, video, or text.

15. The network device of claim 9, wherein the end devices include a sensor device of a first end device and an application of a second end device.

16. A non-transitory computer-readable storage medium storing instructions executable by a processor of a device, which when executed cause the device to:
receive from end devices, monitoring information pertaining to a person, wherein the monitoring information relates to a same health metric;
assign an adaptive weight value to each monitoring information associated with each of the end devices based on profile information for each of the end devices;
aggregate each monitoring information based on the adaptive weight value associated with each of the end devices;
analyze the aggregated monitoring information;
determine, based on the analysis, a health condition of the person;
generate response information that relates to the health condition; and
transmit the response information to a destination device.

17. The non-transitory computer-readable storage medium of claim 16, wherein the profile information includes data indicating operational characteristics and rating information of the end devices.

18. The non-transitory computer-readable storage medium of claim 16, wherein the adaptive weight value indicates a level of quality of each monitoring information.

19. The non-transitory computer-readable storage medium of claim 16, wherein the instructions to aggregate further comprise instructions, which when executed cause the device to:
calculate a weighted average of the same health metric based on the adaptive weight value associated with each monitoring information.

20. The non-transitory computer-readable storage medium of claim 16, wherein the end devices include a sensor device of a first end device and an application of a second end device.
